(19) United States
(12) Patent Application Publication
(10) Pub. No.: US 2007/0054778 A1
(43) Pub. Date: Mar. 8, 2007

(54) APPARATUS AND SYSTEM FOR MEASURING AND COMMUNICATING PHYSICAL ACTIVITY DATA

(76) Inventors: Adrian M. Blanarovich, Chicago, IL (US); Martin V. Seitz, Fox River Grove, IL (US)

Correspondence Address: COOK, ALEX, MCFARRON, MANZO, CUMMINGS & MEHLER LTD, SUITE 2850, 200 WEST ADAMS STREET, CHICAGO, IL, 60606, US

(21) Appl. No.: 11/468,015

(22) Filed: Aug. 29, 2006

Related U.S. Application Data
(60) Provisional application No. 60/712,161, filed on Aug. 29, 2005.

(51) Int. Cl. A63B 71/00; A61B 5/02; A61B 5/103; A61B 5/117

(52) U.S. Cl. 482/8; 600/595; 128/903; 600/500

(57) ABSTRACT

A system is provided for measuring and communicating physical activity data in a multiple user setting. The system generally includes a transmitter for transmitting measured physical activity data. The transmitter produces an addressed signal for the measured physical activity data and transmits the addressed signal to a receiver.

### BACKGROUND OF THE INVENTION

The present invention generally relates to an apparatus and system for measuring and communicating physical activity data and, more specifically, a measurement and communication apparatus and system adapted for use in a multiple user setting.

Traditional apparatuses and systems for measuring and communicating physical activity data generally include a sensor associated with a user's body or in relation to an exercise machine component for sensing the user's movement or the movement of the machine component. Depending on the physical activity to be measured, the data obtained by the sensor is normally transmitted to a computer in order to compute among other things the number of steps taken, cadence, position, velocity, acceleration, distance traveled, calories burned, etc. A monitor is further associated therewith for displaying and indicating such variables.

For example, traditional measurement and communication systems used in measuring and communicating locomotion data for walking or running include a sensor strapped onto the user's foot. U.S. Pat. Nos. 6,513,381 and 6,301,964, Fyfe et al., both for a “Motion Analysis System” describe a device including at least a pair of accelerometers and a tilt sensor typically mounted in fixed relation to a sole of a shoe for extracting kinematic variables including linear and rotational acceleration, velocity and position. These patents do not describe a method for transmitting this data to a controller or display for indicating such variables.

U.S. Pat. No. 6,298,314, Blackadar et al., for a method for “Detecting the Starting and Stopping of Movement of a Person on Foot” describes a method for monitoring movement of a person in locomotion on foot. The method includes mounting a sensor on a user's foot, which generates a signal in response to movement of the person. Data obtained by the sensor is transmitted to a controller for indicating when the person has begun walking or running.

Those skilled in the art will recognize that such traditional systems as described in U.S. Pat. No. 6,298,314 employ transmission means that would create unacceptable data traffic in a multiple user setting such as a marathon environment. This data traffic would create interference with other runners and create inaccurate measurements. Accordingly, it is an object of the invention to provide a portable telemetric measurement system adapted for use in a multiple runner setting.

In another example, traditional measurement and communication systems used in cycling generally include a sensor in relation to at least one of the pedals or the wheels for sensing the movement of the pedal or wheel. The data obtained by the sensor is normally transmitted to a computer in order to compute among other things cadence, speed, acceleration, distance traveled, calories burned, etc. A monitor is further associated therewith for displaying and indicating such variables.

In traditional cyclic measuring systems, the communication means for transmitting data from the sensor to a computer typically involves transmitting data through a communications wire or cable. Nevertheless, communications wires or cables limit the portability of the sensor and/or computer. Accordingly, various wireless communications means have been developed in order to overcome portability difficulties.

U.S. Pat. No. 6,159,130, Torivinen, for a “Measuring Method and Measuring System” describes a measuring method and measuring system suited for measuring the function of at least one organ of a user non-invasively. This patent describes a method of transferring processed measurement data from a data collection unit to a receiver by inductive interaction.

U.S. Pat. No. 6,229,454, Heikkila et al., for a “Telemetric Measuring Method and System” describes a telemetric measuring method in which two or more different variables are measured by different sensors, and the measurement data on each measured variable is transferred by means of telemetric data transmission to the same receiver unit.

U.S. Pat. No. 6,724,299, Takeda et al., for a “Bicycle Data Communication Method and Apparatus” describes a method of communicating data in a bicycle data processing system including the steps of communicating first information from a transmitter to a receiver, wherein the first information has a first rate of change. This method further includes communicating second information from the transmitter to the receiver a plurality of times, wherein the second information has a second rate of change that is greater than the first rate of change.

Nevertheless, each of U.S. Pat. Nos. 6,159,130, 6,229,454 and 6,724,299 describes methods for transmitting an event, and not the calculated data itself. Specifically, for these methods, a transmitter generates a pulse for a heart rate event or a cadence event. This event is transmitted to a receiver, which identifies the pulse and stores it with a reference of time. Accordingly, the transmitter as described in each of these patents transmits data to a receiver each time there is an event. For example, if the user's heart rate is 120 beats per minute (2 Hz), and the cadence is at 120 (2 Hz), 4 pulses are transmitted every second.

Those skilled in the art will recognize that such traditional systems which employ such transmission means would create unacceptable data traffic in a multiple bicycle setting such as a spinning environment. This data traffic would create interference with other cyclists and create inaccurate data measurements. Accordingly, it is an object of the invention to provide a system for measuring and communicating cyclic activity adapted for use in a multiple bicycle setting.

These and other desired benefits of the preferred embodiments, including combinations of features thereof, of the invention will become apparent from the following description. It will be understood, however, that a process or arrangement could come within the scope of the claimed invention without accomplishing each and every one of these desired benefits, including those gleaned from the following description.

### SUMMARY OF THE INVENTION

An apparatus for measuring and communicating physical activity data during cyclic activity of a user is provided. The apparatus includes an accelerometer for measuring cyclic activity and producing a signal in response thereto. A microprocessor is coupled to the accelerometer, wherein the microprocessor determines physical activity data from the accelerometer signal. A display is disposed in relation to the microprocessor for displaying the physical activity data. A housing is provided for accommodating the accelerometer, microprocessor and display. A user securing device affixes the housing to the user.

A system for measuring and communicating physical activity data in a multiple user environment is further provided. The system includes a transmitter for transmitting measured physical activity data. The transmitter produces an addressed signal for the measured physical activity data and transmits the addressed signal to a receiver.

A receiver for communication with a transmitter for receiving physical activity data in a multiple user environment is further provided, wherein the transmitter is adapted for transmitting measured physical activity data and producing an addressed signal in response thereto. The transmitter transmits the addressed signal to a receiving element of the receiver. A display is in relation to the receiving element for displaying physical activity data based on the addressed signal.

A transmitter for communication with a receiver for transmitting physical activity data in a multiple user environment is further provided. The transmitter includes an accelerometer for measuring physical activity and producing a signal in response thereto. A microprocessor is coupled to the accelerometer, wherein the microprocessor provides an address for the signal. A transmission element is coupled to the microprocessor for transmitting the addressed signal.

A receiver for communication with a plurality of transmitters for receiving physical activity data in a multiple user environment is further provided, wherein the transmitters are adapted for transmitting measured physical activity and producing individual addressed signals in response thereto. The transmitter further transmits the individual addressed signal to a receiving element. A display is disposed in relation to the receiving element for displaying physical activity data based on the individual addressed signals.

It should be understood that the present invention includes a number of different aspects or features which may have utility alone and/or in combination with other aspects or features. Accordingly, this summary is not exhaustive identification of each such aspect or feature that is now or may hereafter be claimed, but represents an overview of certain aspects of the present invention to assist in understanding the more detailed description that follows. The scope of the invention is not limited to the specific embodiments described below, but is set forth in the claims now or hereafter filed.

### BRIEF DESCRIPTION OF THE DRAWINGS

FIG. 1 is a perspective view of an apparatus for measuring and communicating physical activity data according to an aspect of the present invention.

FIG. 2 is a top view of the apparatus of FIG. 1.

FIG. 3 is a side view of the apparatus of FIG. 1.

FIG. 4 is an electrical block diagram of power supply circuitry for an apparatus (e.g., the apparatus of FIG. 1) for measuring and communicating physical activity data according to an aspect of the present invention.

FIG. 5 is an electrical block diagram of accelerometer circuitry for an apparatus (e.g., the apparatus of FIG. 1) for measuring and communicating physical activity data according to an aspect of the present invention.

FIG. 6 is a graphical representation of data from an accelerometer mounted on a user's shorts at a cadence of 90 rpm.

FIG. 7 is a graphical representation of data from an accelerometer mounted on a user's shorts at a cadence of 120 rpm.

FIG. 8 is a graphical representation of data from an accelerometer mounted on a user's ankle at a cadence of 60 rpm.

FIG. 9 is an electrical block diagram of microprocessor and display circuitry for an apparatus (e.g., the apparatus of FIG. 1) for measuring and communicating physical activity data according to an aspect of the present invention.

FIG. 10 is a perspective view of a system, including a transmitter and receiver, for measuring and communicating physical activity data according to another aspect of the present invention.

FIG. 11 is a perspective view of a receiver, including a bicycle securing device, for use in a system for measuring and communicating physical activity data according to another aspect of the present invention.

FIG. 12 is a perspective view of a receiver, including a bicycle securing device, for use in a system for measuring and communicating physical activity data according to another aspect of the present invention.

FIG. 13 is a back view of a receiver detailing a bicycle securing device for use in a system for measuring and communicating physical activity data according to another aspect of the present invention.

FIG. 14 is an exploded perspective view of a receiver, including a bicycle securing device, for use in a system for measuring and communicating physical activity data according to another aspect of the present invention.

FIG. 15 is a side view of a receiver, including a bicycle securing device, for use in a system for measuring and communicating physical activity data according to another aspect of the present invention.

FIG. 16 is a back view of a receiver detailing a bicycle securing device for use in a system for measuring and communicating physical activity data according to another aspect of the present invention.

FIG. 17 is a perspective view of a receiver for use in a system for measuring and communicating physical activity data according to another aspect of the present invention.

FIG. 18 is a perspective view of a transmitter for use in a system for measuring and communicating physical activity data according to another aspect of the present invention.

FIG. 19 is a perspective view of a transmitter for use in a system for measuring and communicating physical activity data according to another aspect of the present invention.

FIG. 20 is an electrical block diagram of power supply circuitry for a transmitter (e.g., the transmitter of FIG. 10) for measuring and communicating physical activity data according to an aspect of the present invention.

FIG. 21 is an electrical block diagram of accelerometer circuitry for a transmitter (e.g., the transmitter of FIG. 10) for measuring and communicating physical activity data according to an aspect of the present invention.

FIG. 22 is a graphical representation of data from an accelerometer mounted on a user's ankle at a cadence of 60 rpm.

FIG. 23 is a graphical representation of data from an accelerometer mounted on a user's ankle at a cadence of 90 rpm.

FIG. 24 is a graphical representation of data from an accelerometer mounted on a user's ankle at a cadence of 120 rpm.

FIG. 25 is an electrical block diagram of transmission circuitry for a transmitter (e.g., the transmitter of FIG. 10) for measuring and communicating physical activity data according to an aspect of the present invention.

FIG. 26 an electrical block diagram of circuitry for a receiver (e.g., the receiver of FIG. 10) for measuring and communicating physical activity data according to an aspect of the present invention.

FIG. 27 is a perspective view of a system, including a transmitter and receiver, for measuring and communicating physical activity data according to another aspect of the present invention.

FIG. 28 is a perspective view of a transmitter, including a bicycle securing device, for use in a system for measuring and communicating physical activity data according to another aspect of the present invention.

FIG. 29 is a perspective view of a transmitter, including a bicycle securing device, for use in a system for measuring and communicating physical activity data according to another aspect of the present invention.

FIG. 30 is a perspective view of a transmitter, including a bicycle securing device, for use in a system for measuring and communicating physical activity data according to another aspect of the present invention.

### DETAILED DESCRIPTION OF THE MULTIPLE EMBODIMENTS OF THE PRESENT INVENTION

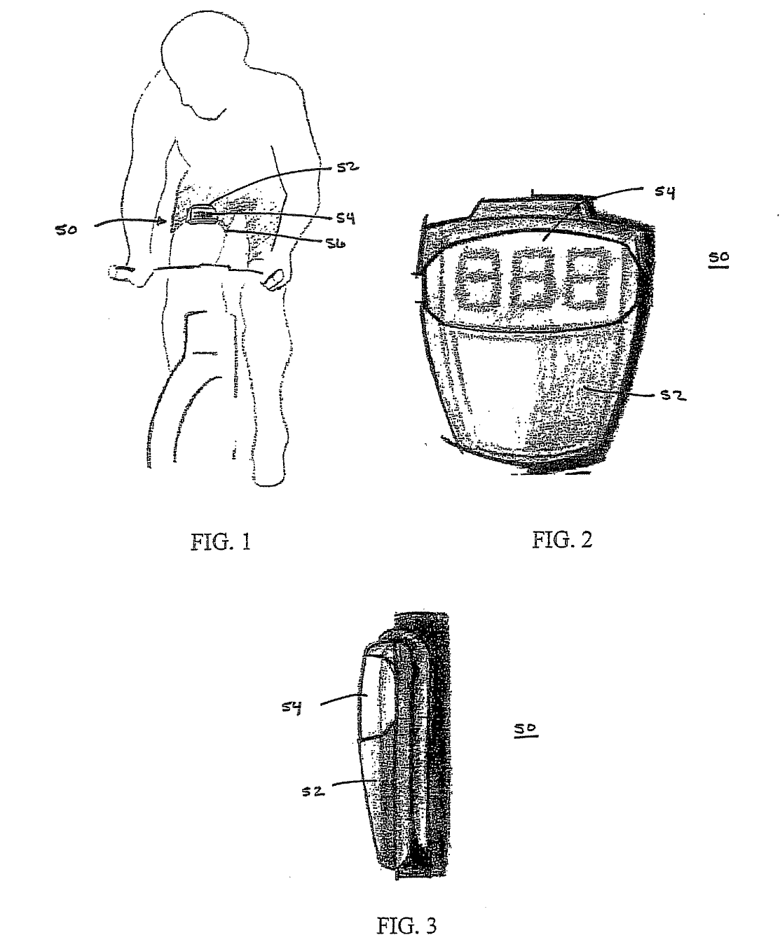

FIGS. 1, 2, and 3 illustrate an embodiment of an apparatus for measuring and communicating physical activity data (e.g., cadence, efficiency of pedal stroke, heartrate, pressure in the foot, speed, acceleration, distance, etc.) in a cycling environment. The measurement and communication apparatus 50 generally includes a plastic housing 52, a display 54, and a user securing device 56 for affixing the system onto the user. The display 54 may be in the form of an LED, LCD, or any other display means. The user securing device 56 may be in the form of a strap as shown in FIG. 3, which secures the measurement and communication apparatus 50 via the plastic housing 52 to the user's leg. In this arrangement, the user is able to easily view the display 54 while cycling. The user securing device 56 may further be in the form of a clip (e.g., which may attach the apparatus to the user's clothing) or any other means for affixing the apparatus onto the user.

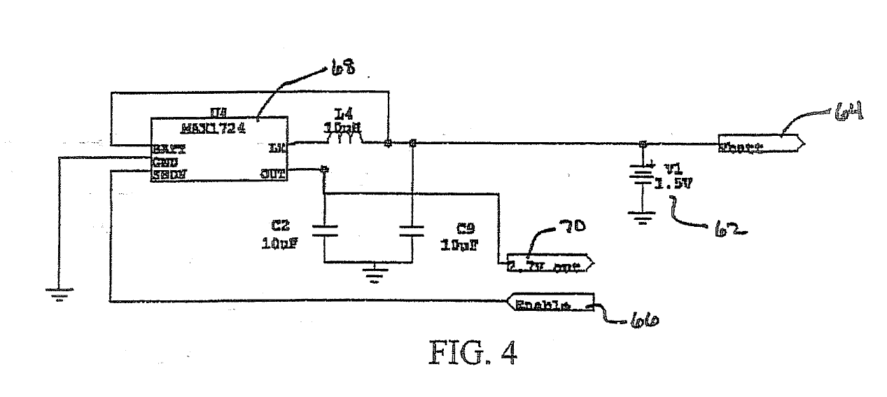

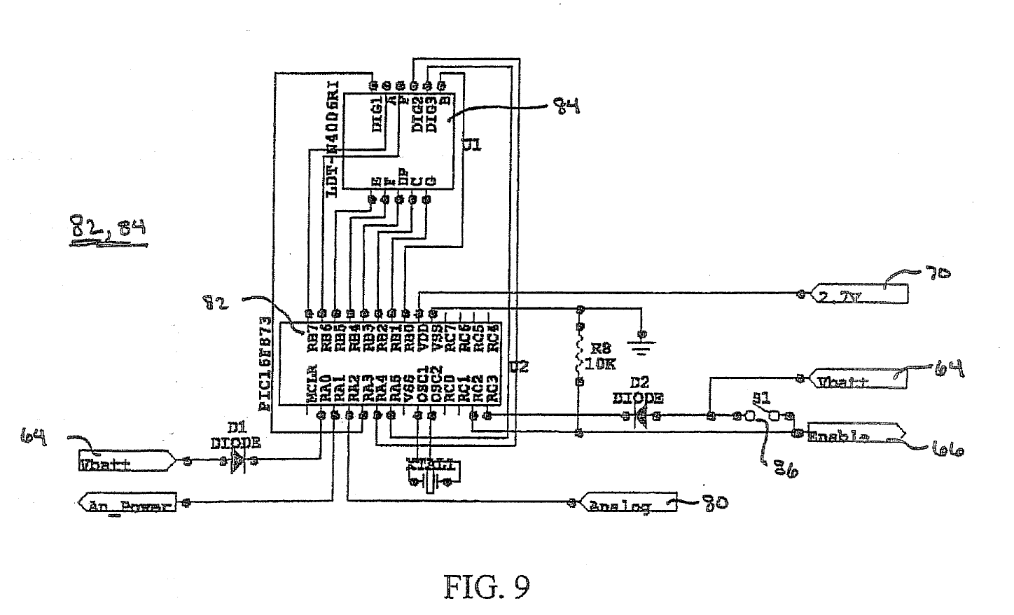

The measurement and communication apparatus 50 of FIGS. 1, 2, and 3 further comprises power supply circuitry 60, accelerometer circuitry 72, a microprocessor 82, and display circuitry 84 as illustrated in the circuit diagrams of FIGS. 4, 5, and 9. As shown in FIG. 4, the apparatus 50 is generally powered by power supply circuitry 60. The power supply circuitry 60 includes a power source generally in the form of a battery 62. The battery 62 generally supplies 1.5V to the apparatus at 64. A switch (not shown) or the microprocessor 82 of FIG. 9 may further be coupled to the power supply circuitry at 66 in order to enable power to the apparatus 50.

In order to allow for another power supply at a higher voltage, the battery 62 is coupled to a step-up converter 68 for allowing a higher voltage power to be supplied to the apparatus 50 at 70. The step-up converter 68 is in the form of a step-up DC-DC converter. For example, as illustrated herein a MAX1724 step-up converter manufactured by Maxim Integrated Products may be used. An advantage of this particular step-up converter is that it includes a low 1.5 μA quiescent supply current to ensure high light-load efficiency. This step-up converter further includes noise-reduction circuitry for suppressing electromagnetic interference (EMI) caused by the inductor. In this arrangement, the step-up converter supplies a 2.7V output. It is important to note that other forms of power sources and step-up converters may be used without deviating from the spirit of the invention. For example, two power sources may be used, one supplying 1.5V at 64 and the other supplying 2.7V at 70.

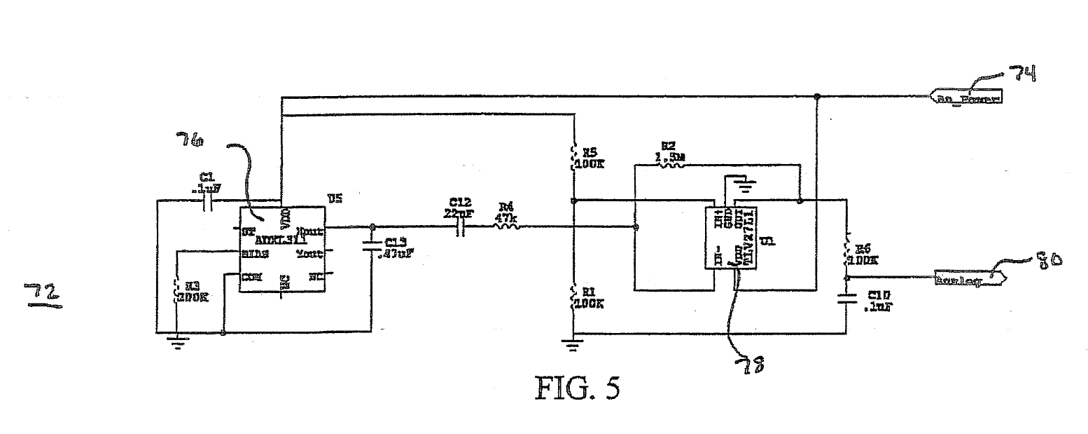

As shown in FIG. 5, the apparatus 50 includes accelerometer circuitry 72. The accelerometer circuitry 72 is generally powered from the output of the power supply available at 70. The accelerometer specifically measures acceleration in the direction of its sensitive axis. An accelerometer that may be used is a dual-axis accelerometer, ADXL311, manufactured by Analog Devices, Inc. This particular accelerometer measures both dynamic acceleration (e.g., vibration) and static acceleration (e.g., gravity). The outputs of the accelerometer are analog voltages proportional to acceleration. It is important to note, however, that other similar accelerometers may be used without deviating from the spirit of the invention.

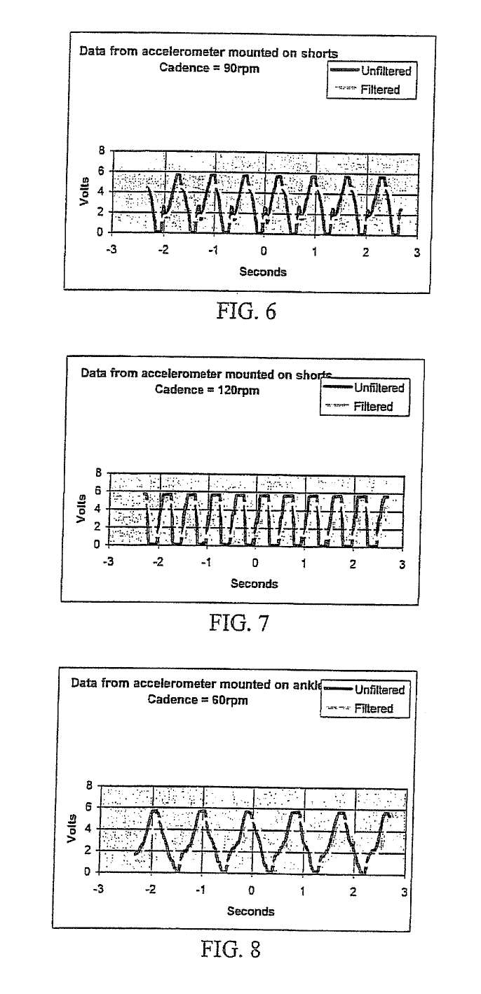

In describing the practical workings of the accelerometer 76, the leg of the person engaged in cycling moves in cyclic motion. The movement of the leg produces analog voltage to an accelerometer 76. In response thereto, these analog voltages are represented by the sine wave output signal as shown in FIGS. 6, 7, and 8. More specifically, if the apparatus 50 is mounted on the leg of the user as shown in FIG. 1, there will be a sine wave output signal produced out of the accelerometer as it moves through two time periods of acceleration (e.g., as the leg moves up, and as it moves down).

Now referring back to FIG. 5, the accelerometer 76 may further be coupled to an operational amplifier 78. The operational amplifier 78 serves to amplify, center and filter the analog voltage outputs of the accelerometer 76. For example, as shown in FIGS. 6, 7, and 8, the sine wave output signals of the accelerometer are filtered accordingly to provide for a more accurate analog voltage output as shown at 80. Although other means for amplifying, centering, and filtering may be used, the operational amplifier used herein may be a TLV27L1 operational amplifier manufactured by Texas Instruments Incorporated.

As shown in FIG. 9, the apparatus 50 includes a microprocessor 82 and display circuitry 84. The microprocessor 82 is powered by the 2.7V output of the step-up converter 70 and 1.5V battery 64 of FIG. 4. The microprocessor 82 may further be coupled to the power supply circuitry at 66 of FIG. 4 in order to enable power to the apparatus 50. A switch 86 may further be coupled to 66 in FIG. 1 in order to allow the user to switch the apparatus on or off. The microprocessor 82 includes an analog/digital converter (A/D converter) for converting the filtered analog output voltage signal 80 from the accelerometer/operational amplifier of FIG. 5 to digital. The microprocessor 82 further determines cadence from the analog output voltage signal 80. In yet another embodiment, the analog output voltage signal 80 is coupled to a comparator for determining the time between each rise of the curve, beyond a select threshold. This time is calculated and compared to a table of possible values, thereby determining velocity or acceleration. Although other microprocessors may also be used, a microprocessor that may be used is a PIC16F873 manufactured by Microchip Technology Inc.

As shown in FIG. 9, the microprocessor 82 is coupled to display circuitry 84 in order to control the display 54 of FIGS. 1, 2, and 3. The display circuitry 84 used herewith is a triple digital display sufficient for displaying the variables associated herewith. Although other display circuitry may also be used, a display processor that may be used is a LDT-N4006R1 manufactured by Lumex Incorporated.

Although the embodiment described in relation to FIGS. 1-9 is described for the cycling environment, it is intended that the measurement and communication apparatus may be adapted for other activities. For example, the measurement and communication apparatus may be adapted to measure other physical activity which involves a user's repetitive motion. For example, the apparatus may be affixed to the user's arm for measuring and communicating a swimmer's or rower's arms' circular motion. Being affixed to the arm, the user will be able to easily view the associated physical data.

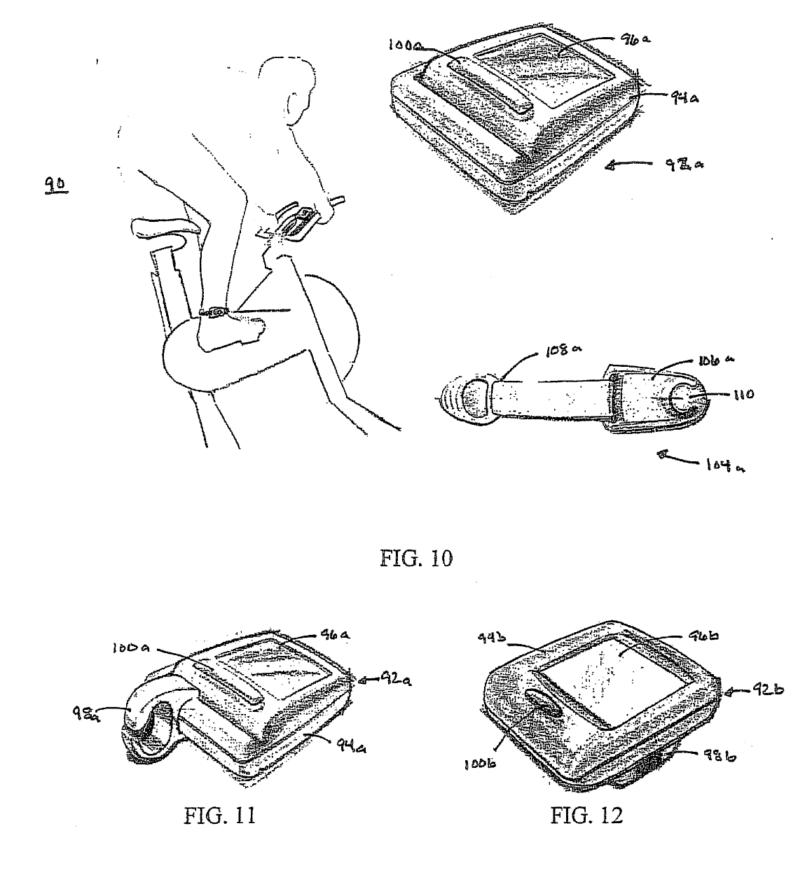

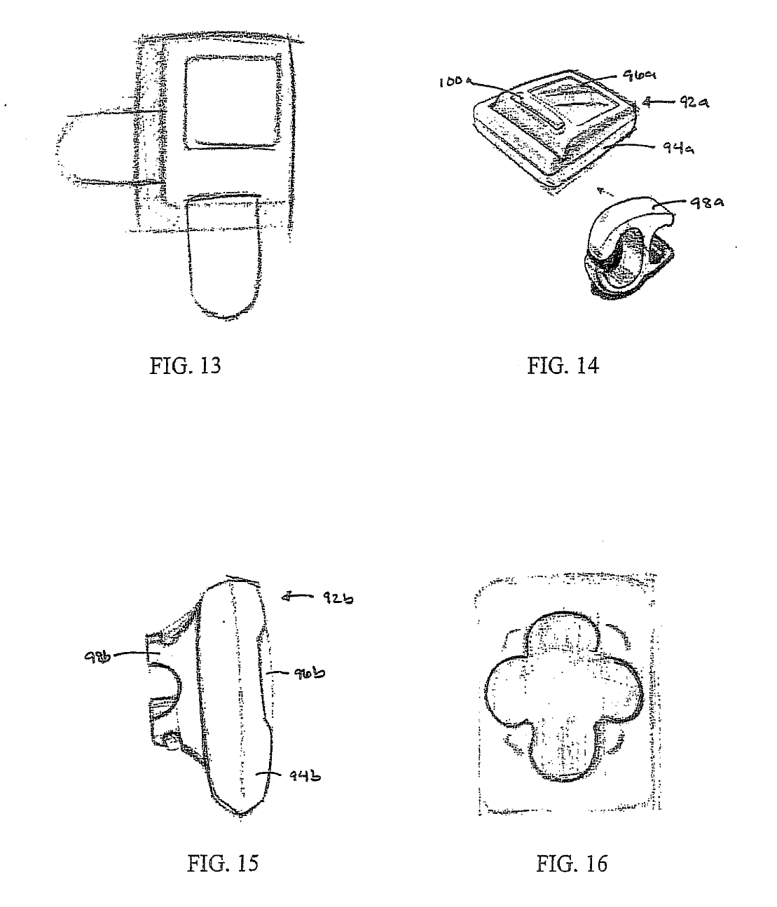

FIG. 10 illustrates a system for measuring and communicating physical activity data (e.g., cadence, efficiency of pedal stroke, heartrate, pressure in the foot, speed, acceleration, distance, etc.) in a cycling environment. The measurement and communication system 90 generally includes a receiver 92 and a transmitter 104. The receiver 92 generally includes a plastic housing 94, a display 96, a bicycle securing device 98 for affixing the receiver onto a bicycle, and a functional button 100. The display 96 may be in the form of an LED, LCD, or any other display means. The functional button 100 resets and switches the receiver 92 on or off. It is important to note that other functional buttons may be incorporated into the receiver 92 for operating or controlling other functions of the receiver 92. For example, a functional button may be included which controls which physical data to display or other suitable operations.

Multiple embodiments of a bicycle securing device 98 are shown in FIG. 11, 12, 14, or 15 which secures the receiver 92 onto the bicycle. The bicycle securing device 98 generally allows the user to easily view the display 96 during cycling. The bicycle securing device 98a may be in the form of a clip as shown in FIGS. 11 and 14, wherein the clip affixes the receiver onto and above the handlebars of the bicycle as shown in FIG. 10. In yet another feature of the securing device 98a, the bicycle securing device 98a is adapted to be removable from the housing of the receiver as specifically shown in FIG. 14. In yet another embodiment as shown in FIGS. 12 and 15, the bicycle securing device 98b is a clip adapted to secure the receiver such that it sits on top of the handlebar. In yet another embodiment, the bicycle securing device 98c is integrated in the housing and includes a slidable member 102 for securing the receiver onto the bicycle. It is important to note that other suitable bicycle securing devices may be used. For example, the securing device may be a strap or any other suitable means which affixes the receiver onto any part of the bicycle.

The transmitter 104 generally includes transmission element 106, a user securing device 108, and an accelerometer 110. The user securing device 108 may further be in the form of a clip (e.g., which may attach the device to the user's ankle, knee, or clothing) or any other means for affixing the transmitter 104 onto any part of the user or any thing or article of clothing associated therewith.

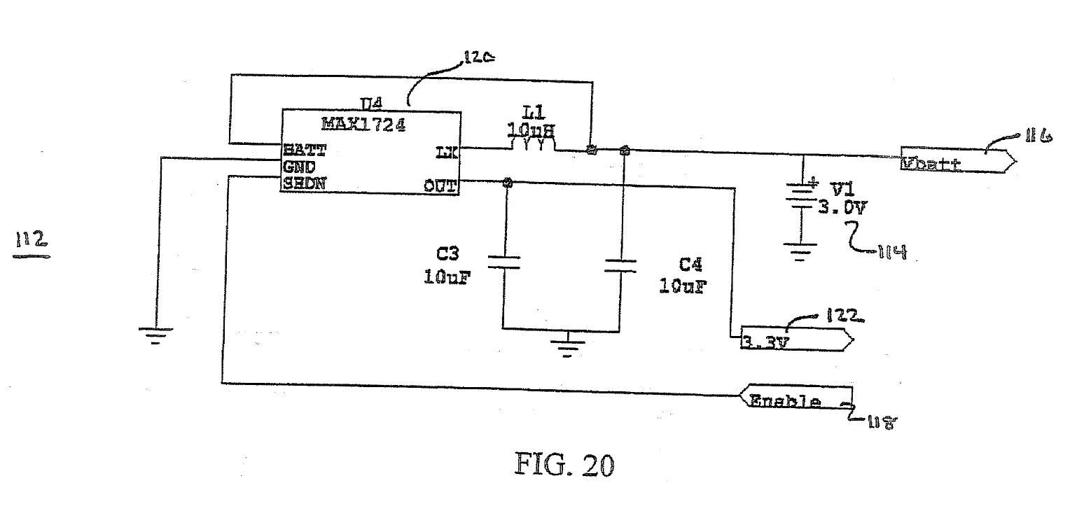

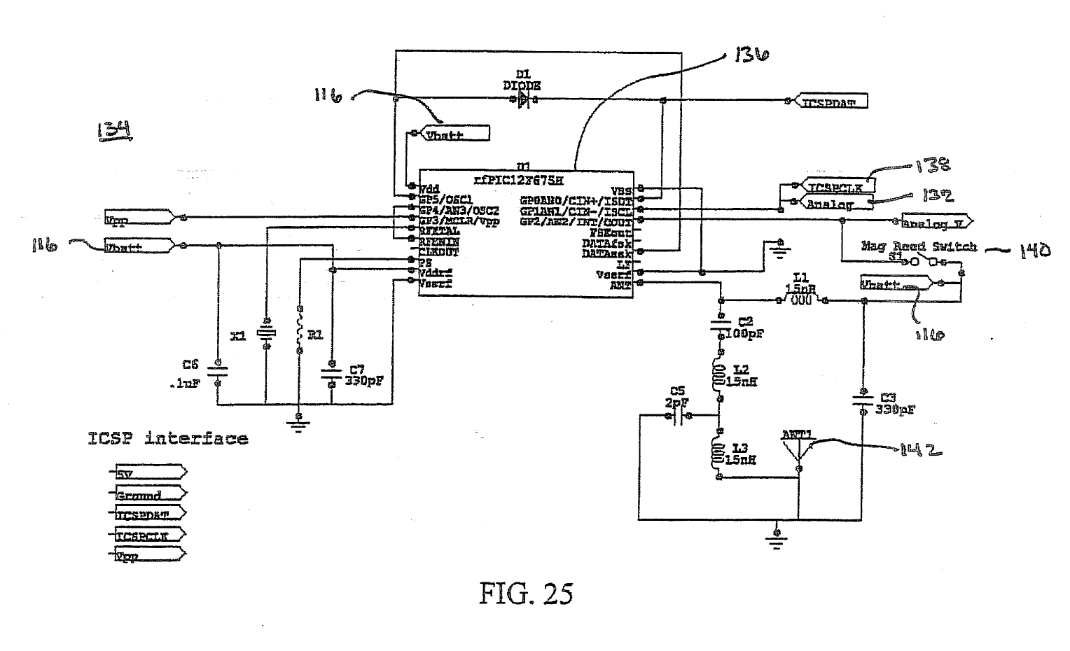

The transmitter 104 of FIGS. 10, 18 and 19 further comprises power supply circuitry 112, accelerometer circuitry 124, and transmission circuitry 134. As shown in FIG. 20, the transmitter 104 is generally powered by power supply circuitry 112. The power supply circuitry 112 includes a power source generally in the form of a battery 114. The battery 114 generally supplies 3.0V to the transmitter at 116. A magnetic reed switch is further shown at 140 in FIG. 25 which will activate the transmitter 104 when it is associated with a magnetic source. This magnetic source may be in the receiver 92. Accordingly, to activate the device the user may briefly place the transmitter 104 in contact with the receiver 92. In this embodiment, the transmitter is further deactivated when it is inactive for several minutes (e.g., the accelerometer does not produce a voltage associated therewith). Other switches may further be implemented in order to activate or deactivate the transmitter 104.

In order to allow for another power supply at a higher voltage, the battery 114 is coupled to a step-up converter 120 for allowing a higher voltage power to be supplied to the transmitter at 122. The step-up converter 122 is in the form of a step-up DC-DC converter. For example, as illustrated herein a MAX1724 step-up converter manufactured by Maxim Integrated Products may be used. An advantage of this particular step-up converter is that it includes a low 1.5 μA quiescent supply current to ensure high light-load efficiency. This step-up converter further includes noise-reduction circuitry for suppressing electromagnetic interference (EMI) caused by the inductor. In this arrangement, the step-up converter supplies a 3.3V output. Other forms of power sources and step-up converters may be used without deviating from the spirit of the invention. For example, two power sources may be used, one supplying 1.5V at 116 and the other supplying 2.7V at 122.

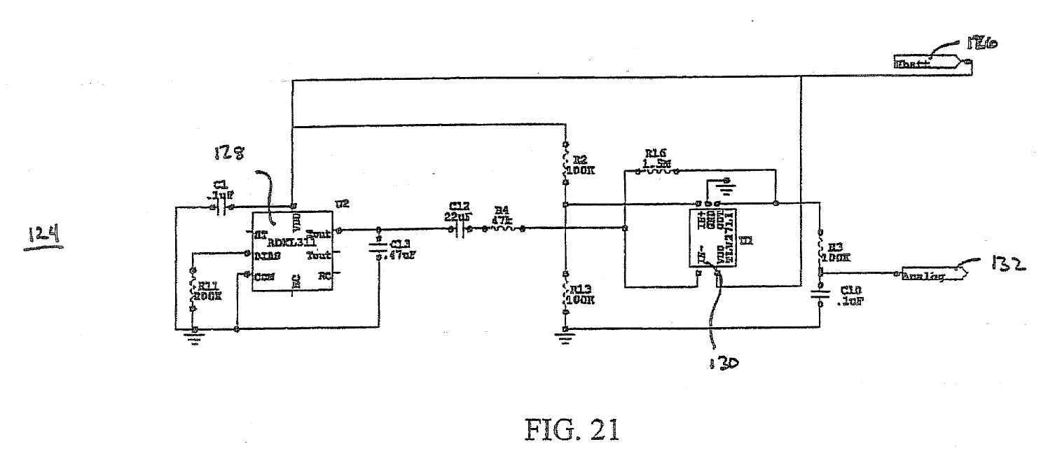

As shown in FIG. 21, the transmitter 104 includes accelerometer circuitry 124. The accelerometer circuitry 124 is generally powered using voltage available from the power supply. The accelerometer specifically measures acceleration in the direction of its sensitive axis. The accelerometer used herein may be a dual-axis accelerometer, ADXL311, manufactured by Analog Devices, Inc. This particular accelerometer measures both dynamic acceleration (e.g., vibration) and static acceleration (e.g., gravity). The outputs of the accelerometer are analog voltages proportional to acceleration. It is important to note, however, that other similar accelerometers may be used without deviating from the spirit of the invention.

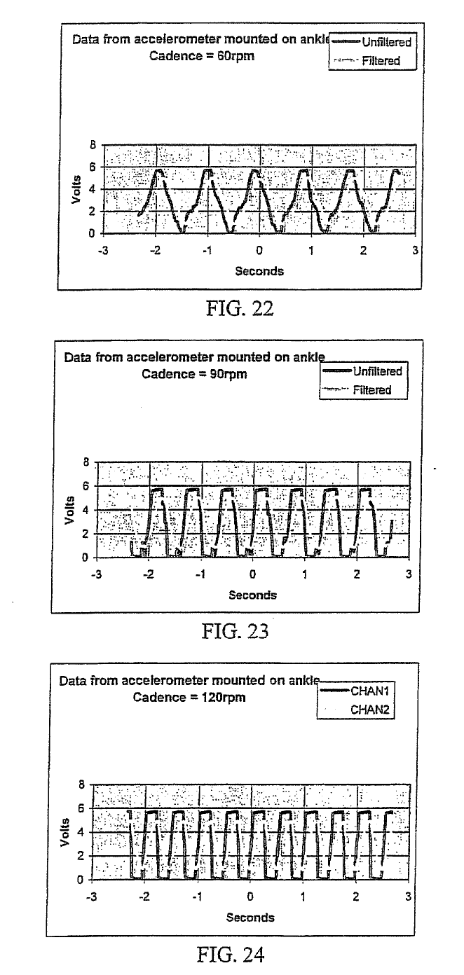

In describing the practical workings of the accelerometer 128, the leg of the person engaged in cycling moves in cyclic motion. The movement of the leg produces analog voltage to an accelerometer 128. In response thereto, these analog voltages are represented by the sine wave output signal as shown in FIGS. 22, 23, and 24. More specifically, if the transmitter 104 is mounted on the ankle of the user as shown in FIG. 10, there will be a sine wave output signal produced out of the accelerometer as it moves through two time periods of acceleration, as the leg moves up, and as it moves down.

Referring back to FIG. 21, the accelerometer 128 may further be coupled to an operational amplifier 130. The operational amplifier 130 serves to amplify, center and filter the analog voltage outputs of the accelerometer 128. For example, as shown in FIGS. 22, 23, and 24, the sine wave output signals of the accelerometer are filtered accordingly to provide for a more accurate analog voltage output as shown at 132. Although other means for amplifying, centering, and filtering may also be used, the operational amplifier used herein may be a TLV27L1 operational amplifier manufactured by Texas Instruments Incorporated.

Now referring to FIG. 25, the analog voltage output as shown at 132 is then transmitted to transmission circuitry 134. Transmission circuitry 134 generally includes a microprocessor/transmitter 136. In this embodiment, the microprocessor/transmitter may be a rfPIC12F675H microchip manufactured by Microchip Technology Inc. This particular microchip includes reference clock, microprocessor, and transmission capability. It is important to note that other suitable microprocessors or transmitters may be used. For example, the microprocessor and transmitter may be separate elements.

Microprocessor/transmitter is powered at by the output of the battery 114 of FIG. 20 at 116. The microprocessor/transmitter 136 converts the incoming analog signal 132 of the accelerometer from FIG. 21 to a digital signal and determines cadence based on a clock input 138. The analog signal 132 may further sent through a comparator in order to determine the time between each rise of the curve beyond a select threshold. This physical activity data is formatted by the microprocessor/transmitter 136 and a unique address/identifier is added to the preamble of the physical activity data. For example, a 2 byte address or identifier may be added to the data. The unique identifier may be preprogrammed or programmable in either the transmitter or receiver. In an example of a preprogrammed transmitter, the unique identifier may be programmed by the manufacturer for use with an particular unique receiver.

The microprocessor/transmitter 136 sends the addressed physical activity data via a modulated radio frequency signal with antenna 142. For example, among other ways, the transmission may be in the form of amplitude shift keying (ASK) or frequency shift keying (FSK) methods. Moreover, the transmitter may transmit in an unlicensed radio frequency band at a suitable speed for minimizing interference from other such devices or any other electronic device used in proximity to current embodiment system (e.g., 900 MHz, 2.4 GHz, 5.8 GHz, etc). In this way, the calculated data itself is transmitted via one group pulses via serial data. In this particular embodiment, addressed physical data is transmitted at approximately 3 bytes once every 1 to 2 seconds, thereby creating less traffic in a multiple user environment.

In yet another embodiment, in order to minimize interference among multiple users in a multiple user environment (e.g., a spin cycling room), the time interval between successive data transmission signals are generated randomly. This random transmission signal may be calculated by the transmitter at activation of the device or during initial set-up.

The receiver 104 includes a receiving element for receiving the physical activity data, a microprocessor 144 and display circuitry 146. The microprocessor 144 is powered by a power source as shown at 148. The power source is shown to be a 3.3V battery although other suitable power sources may be implemented. A switch 150 may further be coupled to the microprocessor 144 in order to allow the user to switch the receiver 104 on or off. Although other microprocessors may be used, the microprocessor used herein is a PIC16F873 manufactured by Microchip Technology Inc.

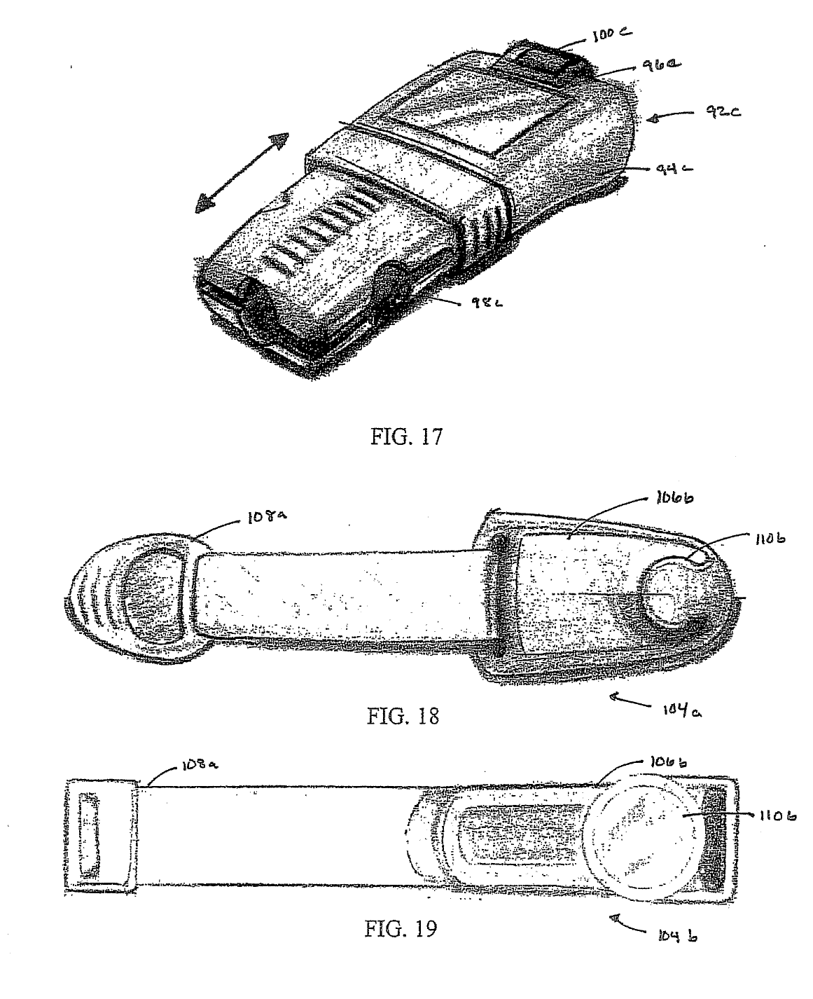

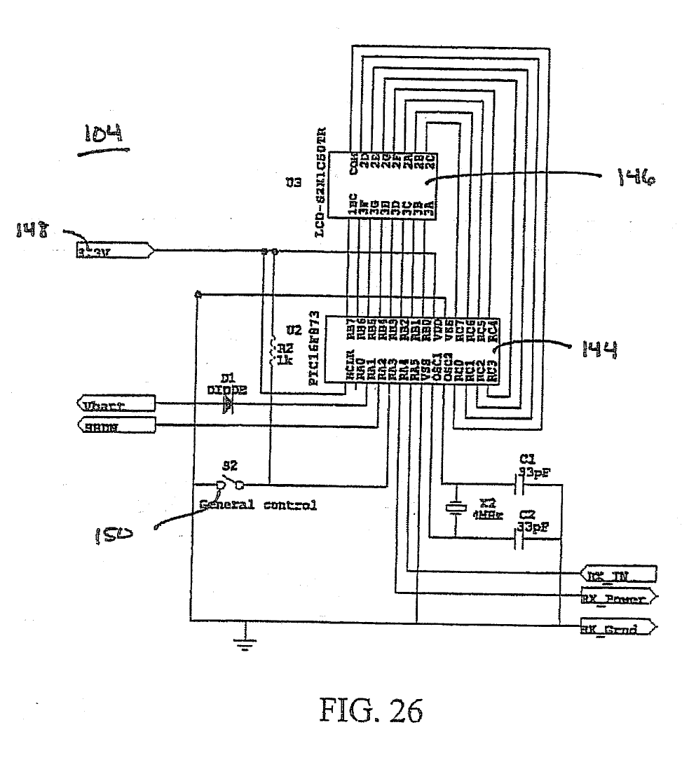

As shown in FIG. 26, the microprocessor 144 is coupled to display circuitry 146 in order to control the display 96 of FIGS. 10-12, 14, 15, and 17. The display circuitry 146 used herewith is a triple digital display sufficient for displaying the variables associated herewith. Although other display circuitry may be used, the display processor used herein may be a LDT-N4006R1 manufactured by Lumex Incorporated.

In yet another embodiment, the display may include a brightness control means on the LCD/LED in order to control such depending on the lighting of the environment. The receiver may also have a means for allowing the receiver to learn the unique address or identifier code of the transmitter. For example, the address or identifier code may be stored in electrically erasable programmable read-only memory (EEPROM). The receiver may further include memory for storing historical physical data. In this embodiment, the microprocessor may be adapted to conFigure, analyze, and sort such data.

In yet another embodiment, another receiver may be implemented in the system. In such an arrangement, the other receiver may receive more than one transmission signal from multiple transceivers. This is desirable in a multiple user setting when there is a trainer, instructor, coach, or the like who is monitoring the progress of multiple users. This is desirable for the instructor for providing feedback to a user which is often unobtainable by current cycling or spinning measurement systems. Because of the unique address of each transceiver, the instructor would be able to identify each user. In yet another embodiment, the receiver may further include memory for storing historical physical data for multiple users. In this embodiment, the microprocessor may be adapted to conFigure, analyze, and sort such data.

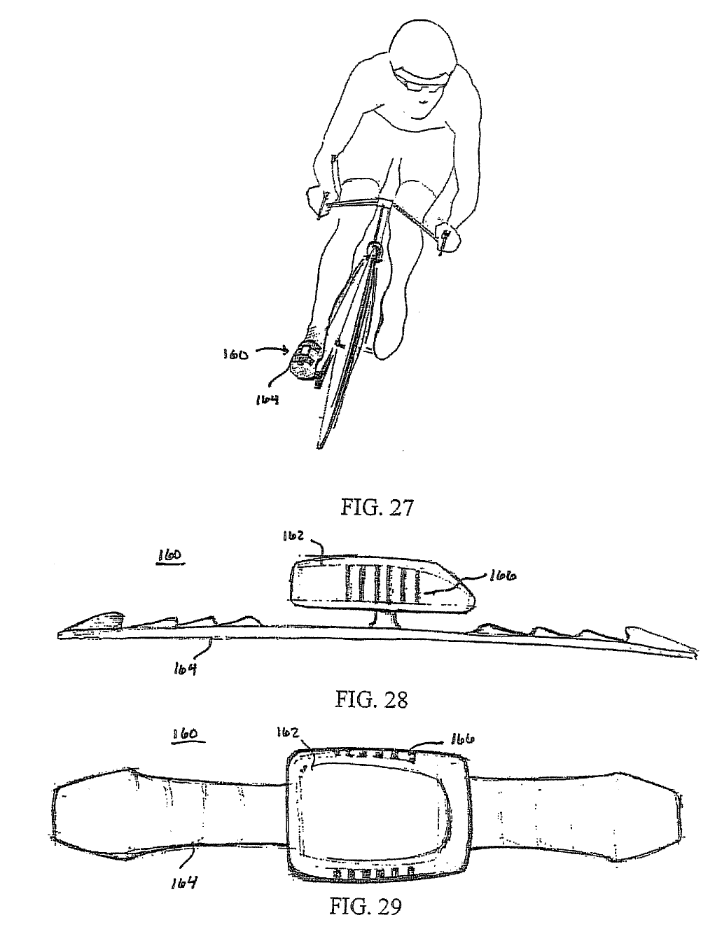

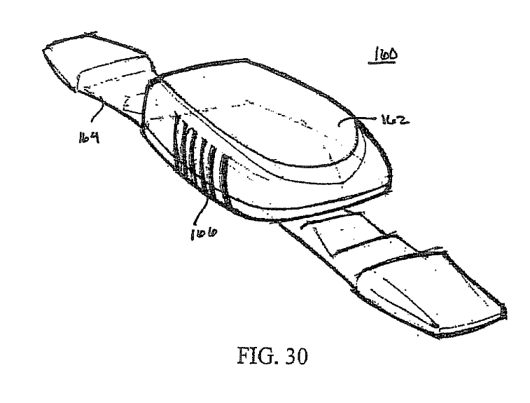

FIGS. 27-30 illustrate yet another embodiment of a transmitter 160 for use in a system for measuring and communicating physical activity data (e.g., cadence, efficiency of pedal stroke, heartrate, pressure in the foot, speed, acceleration, distance, etc.) in a cycling environment. As shown in FIG. 27, the transmitter 160 is secured on the user's foot or shoe. The transmitter 160 generally includes a transmission element 162, a user securing device 164, and an accelerometer 166. The user securing device 108 may further be in the form of a clip (e.g., which may attach the device to the user's ankle, knee, or clothing) or any other means for affixing the apparatus onto any part of the user or any thing or article of clothing associated therewith. The transmitter 160 may be used in conjunction with the receiver as described in the previous embodiments to form a system for measuring and communicating physical activity data.

Although the embodiments described herein are described for the cycling environment, it is intended that the measurement and communication system may be adapted for other activities. For example, the measurement and communication system may be adapted to measure other physical activity which involves a user's repetitive motion (e.g., running, walking, swimming, rowing, or other such activity). For example, the transmitter may be affixed to the user's arm for measuring and communicating a swimmer's or rower's arms' circular motion. The receiver may be also affixed to the wrist (e.g., watch monitor) such that the user will be able to easily view the associated physical data.

While this invention has been described with reference to certain illustrative aspects, it will be understood that this description shall not be construed in a limiting sense. Rather, various changes and modifications that will be apparent to those of skill in the art can be made to the illustrative embodiments without departing from the true spirit and scope of the invention, which is embodied in the appended claims.

1. An apparatus for measuring and communicating physical activity data during cyclic activity of a user, comprising:
an accelerometer for measuring cyclic activity, said accelerometer producing a signal in response thereto,
a microprocessor coupled to said accelerometer, said microprocessor determining physical activity data from said accelerometer signal,
a display coupled to said microprocessor for displaying the physical activity data,
a housing for accommodating said accelerometer, microprocessor and display, and
a user securing device for affixing said housing to the user.

2. The apparatus of claim 1, wherein the user securing device is adapted to affix the housing onto the user's leg.

3. The apparatus of claim 1, wherein the microprocessor is adapted to measure physical activity data selected from the group consisting of cadence, efficiency of pedal stroke, heartrate, pressure in the foot, distance, velocity, and acceleration.

4. A system for measuring and communicating physical activity data in a multiple user environment, comprising:
a transmitter for transmitting measured physical activity data, said transmitter producing an addressed signal for said measured physical activity data and transmitting said addressed signal, and
a receiver for receiving said addressed signal.

5. The system of claim 4, further including a magnetic switch for activating or deactivating either the transmitter or receiver.

6. The system of claim 4, wherein the transmitter includes an accelerometer for measuring the physical activity data.

7. The system of claim 4, wherein the transmitter includes a reference clock for time stamping the measured physical activity data being transmitted.

8. The system of claim 6, wherein the measured physical activity data is cyclic activity.

9. The system of claim 4, wherein the addressed signal includes a unique address for identifying the transmitter.

10. The system of claim 4, wherein the measured physical activity data is transmitted via radio frequency.

11. The system of claim 1, wherein the measured physical activity data is transmitted using a transmission method selected from the group consisting of amplitude shift keying and frequency shift keying.

12. The system of claim 4, wherein the physical activity data is selected from the group consisting of cadence, efficiency of pedal stroke, heartrate, pressure in the foot, distance, velocity, and acceleration.

13. The system of claim 4, wherein the receiver further includes memory for storing the physical activity data.

14. A receiver for communication with a transmitter for receiving physical activity data in a multiple user environment, said transmitter adapted for transmitting measured physical activity data, said transmitter producing an addressed signal for the measured physical activity data and transmitting said addressed signal, comprising:
a receiving element for receiving said addressed signal transmitted by said transmitter, and
a display in relation to said receiving element for displaying physical activity data based on said addressed signal.

15. The receiver of claim 14 further comprising a bicycle securing device for affixing the receiver onto a bicycle.

16. The receiver of claim 14 further including a magnetic switch for activating or deactivating the receiver.

17. The receiver of claim 14, wherein the measured physical activity data is cyclic activity.

18. The receiver of claim 14, further including memory for storing the physical activity data.

19. A transmitter for communication with a receiver for transmitting physical activity data in a multiple user environment, comprising
an accelerometer for measuring physical activity, said accelerometer producing a signal in response thereto,
a microprocessor coupled to said accelerometer, said microprocessor addressing said signal, and
a transmission element coupled to said microprocessor for transmitting said addressed signal.

20. The transmitter of claim 19, further including a magnetic switch for activating or deactivating either the transmitter.

21. The transmitter of claim 19, further including a reference clock for time stamping the measured physical activity data being transmitted.

22. The transmitter of claim 19, wherein the addressed signal includes unique address for identifying the transmitter.

23. The transmitter of claim 19, wherein the measured physical activity data is cyclic activity.

24. The transmitter of claim 19, wherein the measured physical activity data is transmitted via radio frequency.

25. The transmitter of claim 19, wherein the measured physical activity data is transmitted using a transmission method selected from the group consisting of amplitude shift keying and frequency shift keying.

26. The transmitter of claim 19, wherein the physical activity data is selected from the group consisting of cadence, efficiency of pedal stroke, heartrate, pressure in the foot, distance, velocity, and acceleration.

27. A receiver for communication with a plurality of transmitters for receiving physical activity data in a multiple user environment, said transmitters adapted for transmitting measured physical activity data, said transmitters producing individual addressed signals for said measure physical activity data and transmitting said individual addressed signals, comprising:
a receiving element for receiving said individual addressed signals transmitted by said transmitter, and
a display in relation to said receiving element for displaying physical activity data based on said individual addressed signals.

28. The receiver of claim 27, wherein the measured physical activity data is cyclic activity.

29. The receiver of claim 27, further including a memory for storing the physical activity data.
